(19) United States
(12) Patent Application Publication
(10) Pub. No.: US 2026/0152800 A1
(43) Pub. Date: Jun. 4, 2026

(54) BIOMARKER BASED DIAGNOSIS AND TREATMENT OF MYELOPROLIFERATIVE NEOPLASMS

(71) Applicant: UNIVERSITY HEALTH NETWORK, Toronto (CA)

(72) Inventors: John DICK, Toronto (CA); Andy ZENG, Toronto (CA); Jessie MEDEIROS, Toronto (CA); Vikas GUPTA, Toronto (CA); Jean WANG, Toronto (CA)

(21) Appl. No.: 19/126,609

(22) PCT Filed: Nov. 1, 2023
(86) PCT No.: PCT/CA2023/051460

§ 371 Date: May 1, 2025

Related U.S. Application Data
(60) Provisional application No. 63/421,842, filed on Nov. 2, 2022.

(51) Int. Cl. C12Q 1/6827; C12Q 1/6886; G01N 33/68; G16H 50/30

(52) U.S. Cl. CPC C12Q 1/6886; G16B 25/10; C12Q 2600/118; C12Q 2600/158

(57) ABSTRACT

There is described herein a method of prognosing or classifying a subject with a Myeloproliferative Neoplasm (MPN) comprising: (a) determining the expression level of at least 10 genes in a test sample from the subject selected from the group consisting of SPP1, CEACAM6, GJA1, IGSF10, IGFBP2, COL4A5, LYVE1, MTIE, EMP1, XIST, DLK1, TPSAB1, TIMP3, CLC, MS4A1, ENKUR, ALOX12, KNDC1, HLA-DQB1, GAS2, CLEC2L, BEND2, CDH7, and NT5E; and (b) comparing expression of the at least 10 genes in the test sample with reference expression levels of the at least 10 genes from control samples from a reference cohort of patients; wherein a difference or similarity in the expression of the at least 10 genes in the test sample and the reference expression levels is used to prognose or classify the subject with MPN into a low risk group or a high risk group for worse survival.

### RELATED APPLICATIONS

This application claims priority to U.S. Provisional Application No. 63/421,842, filed on Nov. 2, 2022, which is hereby incorporated by reference in its entirety.

### FIELD OF THE INVENTION

The invention relates to the treatment of Myeloproliferative Neoplasms (MPN) and more particularly to biomarkers that assist therewith.

### BACKGROUND OF THE INVENTION

Myelofibrosis (MF) is a myeloproliferative neoplasm (MPN) with survival outcomes ranging from months to years and variable risk of transformation to acute myeloid leukemia (AML). Allogeneic bone marrow transplantation (BMT) can be curative but is associated with high treatment related morbidity and mortality, therefore accurate risk stratification is important to guide clinical decision making in MF. Current risk prediction models use clinical and/or genomic features but do not consider the properties of the disease-driving stem cell population.

### SUMMARY OF THE INVENTION

In an aspect there is provided a method of prognosing or classifying a subject with a Myeloproliferative Neoplasm (MPN) comprising:
(a) determining the expression level of at least 10 genes in a test sample from the subject selected from the group consisting of SPP1, CEACAM6, GJA1, IGSF10, IGFBP2, COL4A5, LYVE1, MT1E, EMP1, XIST, DLK1, TPSAB1, TIMP3, CLC, MS4A1, ENKUR, ALOX12, KNDC1, HLA-DQB1, GAS2, CLEC2L, BEND2, CDH7, and NT5E; and
(b) comparing expression of the at least 10 genes in the test sample with reference expression levels of the at least 10 genes from control samples from a reference cohort of patients;
wherein a difference or similarity in the expression of the at least 10 genes in the test sample and the reference expression levels is used to prognose or classify the subject with MPN into a low risk group or a high risk group for worse survival.

In a further aspect there is provided a method of determining the risk of transformation of a Myeloproliferative Neoplasm (MPN) to acute myeloid leukemia, in a subject with MPN, comprising:
(a) determining the expression level of at least 5 genes in a test sample from the subject selected from the group consisting of SPP1, TPSAB1, COL4A5, CEACAM6, IGFBP2, EMP1, DLK1, IGSF10, HLA-DQB1, KNDC1, CLEC2L, CDH7, or ENKUR; and
(b) comparing expression of the at least 5 genes in the test sample with reference expression levels of the at least 5 genes from control samples from a reference cohort of patients;
wherein a difference or similarity in the expression of the at least 5 genes in the test sample and the reference expression levels is used to prognose or classify the subject with MPN into a low risk group or a high risk group for transformation to secondary acute myeloid leukemia (sAML).

In an aspect there is provided a computer program product for use in conjunction with a computer having a processor and a memory connected to the processor, the computer program product comprising a computer readable storage medium having a computer mechanism encoded thereon, wherein the computer program mechanism may be loaded into the memory of the computer and cause the computer to carry out the method described herein.

### BRIEF DESCRIPTION OF FIGURES

These and other features of the preferred embodiments of the invention will become more apparent in the following detailed description in which reference is made to the appended drawings wherein:

FIG. 1: Introduction to Mpn and Mf.

FIG. 2: Current risk assessment tools in MPN.

FIG. 3: Approach: PMCC Clinical Cohort-Assembly.

FIG. 4: Approach: PMCC Clinical Cohort-Clinical Data Collection.

FIG. 5: Approach: PMCC Clinical Cohort-RNA sequencing.

FIG. 6: Approach: Prognostic Signature Training. 1. Setup.

FIG. 7: Approach: Prognostic Signature Training. 2. Scheme.

FIGS. 8-12: Approach: Prognostic Signature Training. 3. Models generation and testing.

FIG. 13: Approach: Prognostic Signature Training. 4. MPN24 Gene Expression Signature.

FIG. 14: Evaluate. MPN24 in Training and Test Sets.

FIG. 15: Evaluate. MPN24 in Test Sets. Multivariable Analysis. 1.

FIG. 16: Evaluate. MPN24 in Test Set. Multivariable Analysis. 2.

FIG. 17: Integrate. MPN24 with DIPSS to generate a 3-tier stratification model in MF. 1.

FIG. 18: Integrate. MPN24 with DIPSS to generate a 3-tier stratification model in MF. 2.

FIG. 19: Evaluate. MPN24 in relation to the MIPSS70 score in MF.

FIG. 20: Approach. Signature Training for Predicting Leukemia Transformation. MPN13.

FIG. 21: Evaluate. MPN13 in Train and Test Sets.

FIG. 22: High correlation of MPN24 and MPN13 when measured by NanoString and RNAseq in the same samples (n=24).

### DETAILED DESCRIPTION

In the following description, numerous specific details are set forth to provide a thorough understanding of the invention. However, it is understood that the invention may be practiced without these specific details.

Applicants used transcriptomic variation corresponding to both intra- and inter-patient heterogeneity among MF stem cells to generate novel gene expression-based scores predictive of survival and leukemic transformation in MF.

To train and validate novel prognostic scores in MF, we identified 358 patients from an MPN registry at the Princess Margaret Cancer Centre (ClinicalTrials.gov Identifier: NCT02760238) from whom peripheral blood (PB) cells were collected near the date of MF diagnosis. All patients were diagnosed with either primary, post-PV, post-ET or pre-fibrotic MF with clinical follow-up of up to 12.2 years. RNA was extracted from unsorted PB mononuclear cells and RNA sequencing (RNAseq) was performed at an average depth of 50 million reads per sample. We randomly split our MF cohort into training (70%; n=250) and test sets (30%; n=108) and utilized a repeated nested cross validation approach together with statistical regression, to generate and assess the performance of models to predict survival within the training set. We tested 36,000 models derived from 36 initial MF-related genesets, ranging from stem-cell specific genesets to the whole transcriptome. The most accurate models by cross validation (median multivariable p-value=6e-5) were produced from our retrospective identification of highly variable genes in single-cell RNAseq data derived from 82,255 Lin-CD 34+ MF stem and progenitor cells across 15 patients (Psaila et al., 2020). Thus, features of intra- and inter-patient heterogeneity among MF stem and progenitor cells proved to be the most relevant for predicting survival. From these features, we derived our final model calculated as the weighted sum of gene expression across 24 genes (termed MPN24).

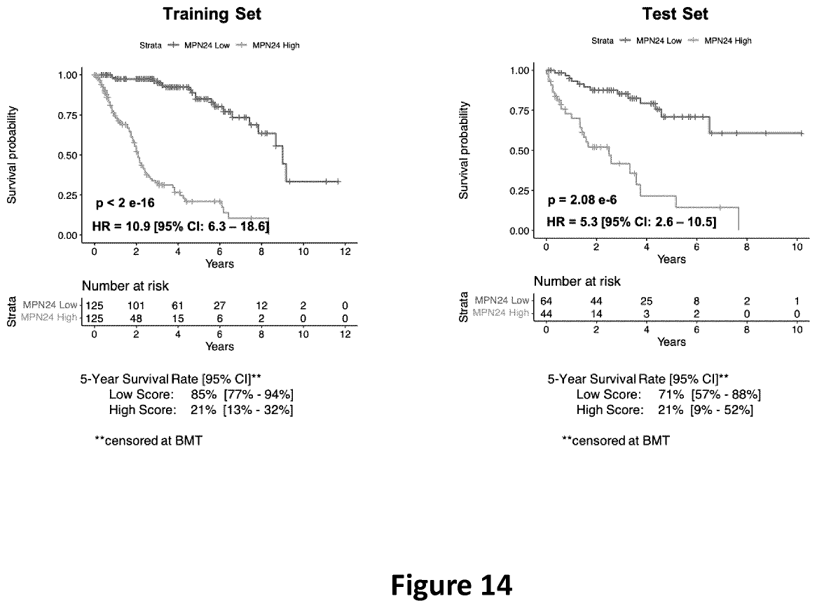

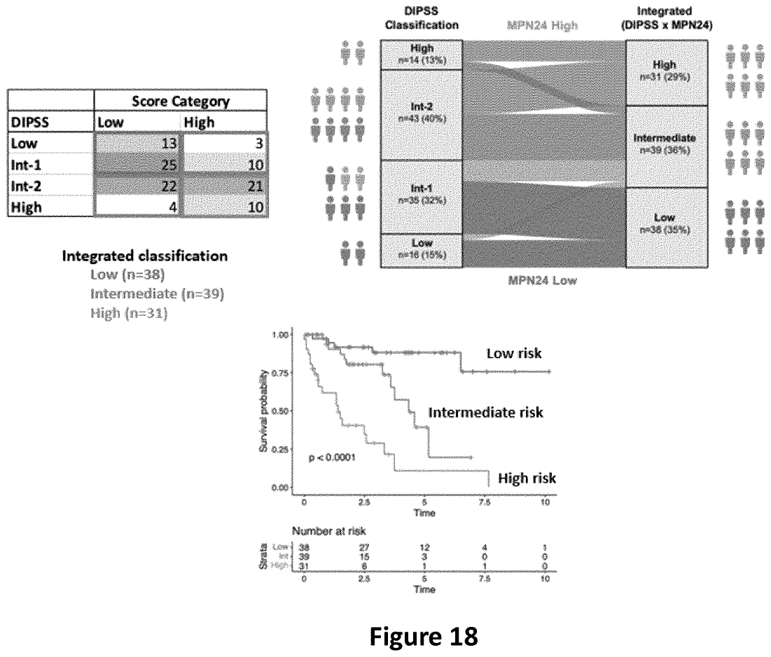

We categorized patients with MPN24 scores above or below the training cohort median as MPN24 high or low, respectively. This model was validated in the test set, with high and low score patients experiencing 5-year survival rates of 71% [95% CI 57-88%] and 21% [95% CI 9%-52%], respectively, when censored at time of BMT (HR=5.3[95 % CI 2.6-10.5]; p=2.1e-6) (FIG. 14; right panel). MPN24 retained independent prognostic value in multivariable analysis incorporating age, sex, DIPSS category, ECOG status, fibrosis grade, constitutional symptoms, and PB blast percentage (adjusted HR=5.7[95 % CI 2.2-14]; p=3 e-4). Importantly, DIPSS classification remained a significant covariate, indicating that MPN24 and DIPSS capture distinct features of disease. We therefore developed a new three-tier classification scheme integrating both DIPSS and MPN24 scores (FIG. 18). Patients classified as low-, intermediate- or high-risk in this new classification scheme experienced 5-year survival rates of 88.2% [95% CI 77.9%-99.9%], 39.3% [95% CI 19.9%-77.7%] and 10.8% [95% CI 2.1%-55.8%], respectively (likelihood ratio test p=1e-8). Further, MPN24 is provides additional resolution for survival for patients classified as intermediate and high risk by MIPSS70. Finally, from the MPN24 genes we derived a 13 gene subscore (MPN13) predictive of leukemic transformation in the training set. Patients in the test set scoring above the 80th percentile from the training set were classified as high risk and the remaining as low risk. Although total leukemic samples were limiting, MPN13 was significantly associated with risk of transformation (p=4.7e-03) with low and high-risk patients experiencing 3-year cumulative incidences of transformation of 5.2% [95% CI 0.2%-10.2%] and 28.6% [95% CI 3.1%-54.0%] respectively, after adjusting for death as a competing risk. These scores have been transferred for testing in independent cohorts on a Nanostring platform and was validated.

In summary, we used transcriptional variation among MF stem and progenitor cells to derive novel gene expression scores predictive of survival and leukemic transformation and developed a new integrated 3-tier model for predicting risk in MF patients.

In an aspect there is provided a method of prognosing or classifying a subject with a Myeloproliferative Neoplasm (MPN) comprising:
(a) determining the expression level of at least 10 genes in a test sample from the subject selected from the group consisting of SPP1, CEACAM6, GJA1, IGSF10, IGFBP2, COL4A5, LYVE1, MT1E, EMP1, XIST, DLK1, TPSAB1, TIMP3, CLC, MS4A1, ENKUR, ALOX12, KNDC1, HLA-DQB1, GAS2, CLEC2L, BEND2, CDH7, and NT5E; and
(b) comparing expression of the at least 10 genes in the test sample with reference expression levels of the at least 10 genes from control samples from a reference cohort of patients;
wherein a difference or similarity in the expression of the at least 10 genes in the test sample and the reference expression levels is used to prognose or classify the subject with MPN into a low risk group or a high risk group for worse survival.

The term “prognosis” as used herein refers to a clinical outcome group such as a worse survival group or a better survival group associated with a disease subtype which is reflected by a reference profile such as a biomarker reference expression profile or reflected by an expression level of the 10 biomarkers disclosed herein. The prognosis provides an indication of disease progression and includes an indication of likelihood of death due to the disease. In one embodiment the clinical outcome class includes a better survival group and a worse survival group.

The term “prognosing or classifying” as used herein means predicting or identifying the clinical outcome group that a subject belongs to according to the subject's similarity to a reference profile or biomarker expression level associated with the prognosis. For example, prognosing or classifying comprises a method or process of determining whether an individual with MPN has a better or worse survival outcome, or grouping an individual with MPN into a better survival group or a worse survival group, or predicting whether or not an individual with MPN will respond to therapy.

The term “subject” as used herein refers to any member of the animal kingdom, preferably a human being and most preferably a human being that has MPN or that is suspected of having MPN.

The term “test sample” as used herein refers to any fluid, cell or tissue sample from a subject which can be assayed for biomarker expression products and/or a reference expression profile, e.g. genes differentially expressed in subjects with MPN according to survival outcome.

The phrase “determining the expression of biomarkers” as used herein refers to determining or quantifying RNA or proteins or protein activities or protein-related metabolites expressed by the biomarkers. The term “RNA” includes mRNA transcripts, and/or specific spliced or other alternative variants of mRNA, including anti-sense products. The term “RNA product of the biomarker” as used herein refers to RNA transcripts transcribed from the biomarkers and/or specific spliced or alternative variants. In the case of “protein”, it refers to proteins translated from the RNA transcripts transcribed from the biomarkers. The term “protein product of the biomarker” refers to proteins translated from RNA products of the biomarkers.

The term “level of expression” or “expression level” as used herein refers to a measurable level of expression of the products of biomarkers, such as, without limitation, the level of micro-RNA, messenger RNA transcript expressed or of a specific exon or other portion of a transcript, the level of proteins or portions thereof expressed of the biomarkers, the number or presence of DNA polymorphisms of the biomarkers, the enzymatic or other activities of the biomarkers, and the level of specific metabolites.

As used herein, the term “control” refers to a specific value or dataset that can be used to prognose or classify the value e.g expression level or reference expression profile obtained from the test sample associated with an outcome class. In one embodiment, a dataset may be obtained from samples from a group of subjects known to have MPN and better survival outcome or known to have MPN and have worse survival outcome or known to have MPN and have benefited from chemotherapy (or intensified chemotherapy) or known to have MPN and not have benefited from chemotherapy (or intensified chemotherapy). The expression data of the biomarkers in the dataset can be used to create a control value that is used in testing samples from new patients. In such an embodiment, the “control” is a predetermined value for the set of biomarkers obtained from MPN patients whose biomarker expression values and survival times are known. Alternatively, the “control” is a predetermined reference profile for the set of biomarkers described herein obtained from patients whose survival times are known.

The term “differentially expressed” or “differential expression” as used herein refers to a difference in the level of expression of the biomarkers that can be assayed by measuring the level of expression of the products of the biomarkers, such as the difference in level of mRNA or a portion thereof expressed. In a preferred embodiment, the difference is statistically significant. The term “difference in the level of expression” refers to an increase or decrease in the measurable expression level of a given biomarker, for example as measured by the amount of mRNA as compared with the measurable expression level of a given biomarker in a control.

The term “better survival” as used herein refers to an increased chance of survival as compared to patients in the “worse survival” group. For example, the biomarkers of the application can prognose or classify patients into a “better survival group”. These patients are at a lower risk of death from the disease.

The term “worse survival” as used herein refers to an increased risk of death as compared to patients in the “better survival” group. For example, biomarkers or genes of the application can prognose or classify patients into a “worse survival group”. These patients are at greater risk of death or adverse reaction from disease, treatment for the disease or other causes.

In some embodiments, the at least 10 genes is at least 11, 12, 13, 14, 15, 16, 17, 18, 19, 20, 21, 22, 23, or 24 genes.

In some embodiments, the at least 10 genes consists of all 24 genes.

In some embodiments, the method further comprises building a subject gene expression (GE) profile from the determined expression of the at least 10 genes.

In some embodiments, the method further comprises obtaining a reference GE profile associated with a prognosis, wherein the subject GE profile and the gene reference expression profile each have values representing the expression level of the at least 10 genes.

In some embodiments, the method further comprises calculating a MPN24 Score comprising the weighted sum expression of the at least 10 genes.

In some embodiments, classification of the subject into a high risk group is based on a high MPN24 Score in reference to the control cohort of MPN patients.

In some embodiments, classification of the subject into a high risk group or low risk group is based on whether the subject MPN24 Score is above or below a predetermined threshold, for example, a mean or preferably median MPN24 Score of the reference cohort.

In some embodiments, determining the GE level comprises use of RNAseq, quantitative PCR or an array.

In some embodiments, determining the GE level comprises use of nanostring.

A person skilled in the art will appreciate that a number of methods can be used to detect or quantify the level of RNA products of the biomarkers within a sample, including arrays, such as microarrays, RT-PCR (including quantitative RT-PCR), nuclease protection assays and Northern blot analyses. For example, biomarkers may be measured using one or more methods and/or tools, including for example, but not limited to, Taqman (Life Technologies, Carlsbad, Calif.), Light-Cycler (Roche Applied Science, Penzberg, Germany), ABI fluidic card (Life Technologies), NanoString. RTM. (NanoString Technologies, Seattle, Wash. and as described in U.S. Pat. No. 7,473,767), NANODROP™ technology (Thermo Fisher Scientific (Wilmington, Del.), fluidic card, and the like. The person of skill in the art will recognize such other formats and tools, which can be commercially available or which can be developed specifically for such analysis. Regarding nanostring specifically, it is also known to use synthetic oligonucleotides as a control in each nanostring cartridge to minimize inter-cartridge batch effects between runs.

In some embodiments, the method further comprises stratifying the patients based on a further criteria.

In some embodiments, the further criteria comprises sex, DIPSS category, ECOG status, fibrosis grade, constitutional symptoms, MIPSS70 category, or PB blast percentage.

DIPSS category features may include age, constitutional symptoms, white blood cell count, hemoglobin, and % blasts in PB blood as previously described.

MIPSS70 category features may include platelet count, bone marrow fibrosis grade, mutations in the following genes: CALR, ASXL1, EZH2, SRSF2, IDH1, IDH2 or U2AF1 and/or karyotype status as previously described.

In some embodiments, the MPN is myelofibrosis (MF).

In some embodiments, the MPN is Polycythemia Vera (PV).

In some embodiments, the MPN is Essential Thrombocythemia (ET).

In some embodiments, the MPN is Chronic Myelogenous Leukemia (CML).

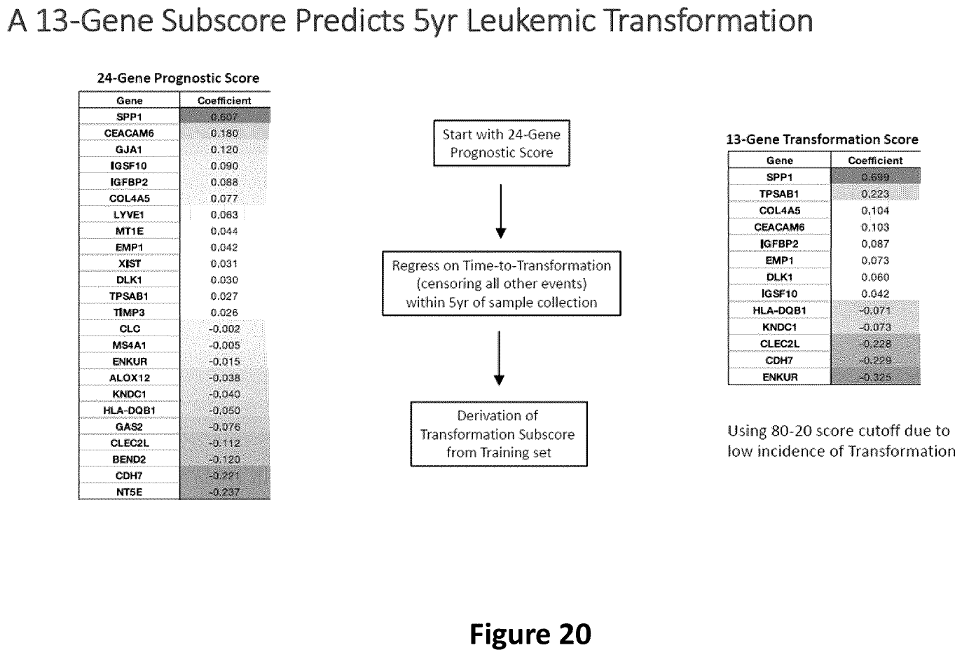

In some embodiments, the at least 10 genes are selected based on the most highly correlated genes and coefficients in FIG. 20.

In some embodiments, the method further comprises treating the subject with more aggressive therapy if the subject has been determined to be in the high risk group for worse survival.

In some embodiments, the more aggressive therapy is bone marrow transplant.

In some embodiments, the more aggressive therapy is adjuvant therapy, intensified chemotherapy or an alternative therapy through enrollment into a clinical trial for a novel therapy.

Regimens for standard vs. intensified chemotherapy are known in the art. Intensified chemotherapy may comprise any chemotherapy that is increased along at least one axis (e.g. dose, duration, frequency, . . . etc.) as compared to standard chemotherapy treatment for a particular cancer type and stage.

In a further aspect there is provided a method of determining the risk of transformation of a Myeloproliferative Neoplasm (MPN) to acute myeloid leukemia, in a subject with MPN, comprising:
(a) determining the expression level of at least 5 genes in a test sample from the subject selected from the group consisting of SPP1, TPSAB1, COL4A5, CEACAM6, IGFBP2, EMP1, DLK1, IGSF10, HLA-DQB1, KNDC1, CLEC2L, CDH7, or ENKUR; and
(b) comparing expression of the at least 5 genes in the test sample with reference expression levels of the at least 5 genes from control samples from a reference cohort of patients;
wherein a difference or similarity in the expression of the at least 5 genes in the test sample and the reference expression levels is used to prognose or classify the subject with MPN into a low risk group or a high risk group for transformation to secondary acute myeloid leukemia (sAML).

In some embodiments, the at least 5 genes is at least 6, 7, 8, 9, 10, 11, 12, or 13 genes.

In some embodiments, the at least 5 genes consists of all 13 genes.

In some embodiments, the method further comprises building a subject gene expression (GE) profile from the determined expression of the at least 5 genes.

In some embodiments, the method further comprises obtaining a reference GE profile associated with a risk of transformation to AML, wherein the subject GE profile and the gene reference expression profile each have values representing the expression level of the at least 5 genes.

In some embodiments, the method further comprises calculating a MPN13 Score comprising the weighted sum expression of the at least 5 genes.

In some embodiments, classification of the subject into a high risk group is based on a high MPN13 Score in reference to the control cohort of MPN patients.

In some embodiments, classification of the subject into a high risk group or low risk group is based on whether the subject MPN13 Score is above or below, respectively, the 50^{th}, 60^{th}, 70, and preferably, 80^{th} percentile MPN13 Score of the reference cohort.

In some embodiments, determining the GE level comprises use of RNAseq, quantitative PCR or an array.

In some embodiments, determining the GE level comprises use of nanostring.

In some embodiments, the method further comprises stratifying the patients based on a further criteria.

In some embodiments, the further criteria comprises sex, DIPSS category, ECOG status, fibrosis grade, constitutional symptoms, MIPSS70 category, or PB blast percentage.

In some embodiments, the MPN is myelofibrosis (MF).

In some embodiments, the MPN is Polycythemia Vera (PV).

In some embodiments, the MPN is Essential Thrombocythemia (ET).

In some embodiments, the MPN is Chronic Myelogenous Leukemia (CML).

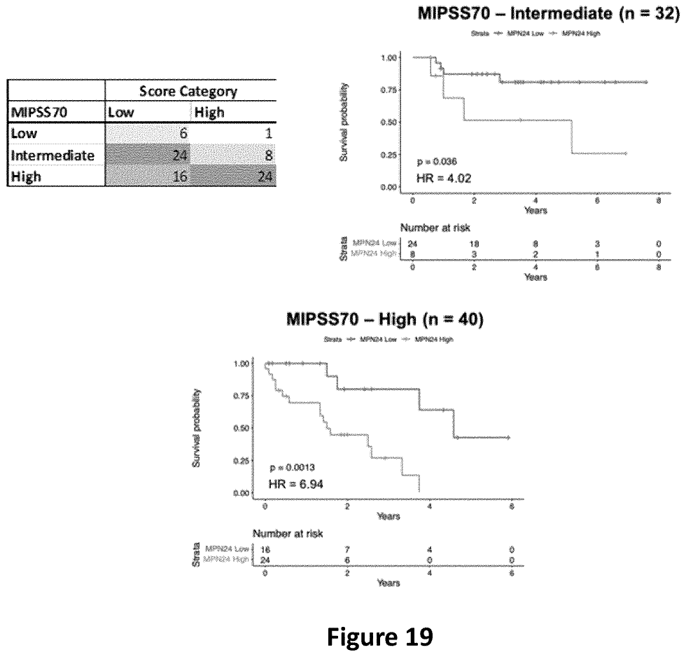

In some embodiments, the at least 10 genes are selected based on the most highly correlated genes based on the coefficients in FIG. 19.

In some embodiments, the method further comprises treating the subject with more aggressive therapy if the subject has been determined to be in the high risk group for transformation to sAML.

In some embodiments, the more aggressive therapy is bone marrow transplant.

In some embodiments, the more aggressive therapy is adjuvant therapy, intensified chemotherapy or an alternative therapy through enrollment into a clinical trial for a novel therapy.

In an aspect there is provided a computer program product for use in conjunction with a computer having a processor and a memory connected to the processor, the computer program product comprising a computer readable storage medium having a computer mechanism encoded thereon, wherein the computer program mechanism may be loaded into the memory of the computer and cause the computer to carry out the method described herein.

In a further aspect, there is provided a kit comprising reagents for detecting the expression of the genes that form a part of the methods described above.

In a further aspect, there is provided an array or nanostring for detecting the expression of the genes that form a part of the methods described above.

The advantages of the present invention are further illustrated by the following examples. The examples and their particular details set forth herein are presented for illustration only and should not be construed as a limitation on the claims of the present invention.

### EXAMPLES

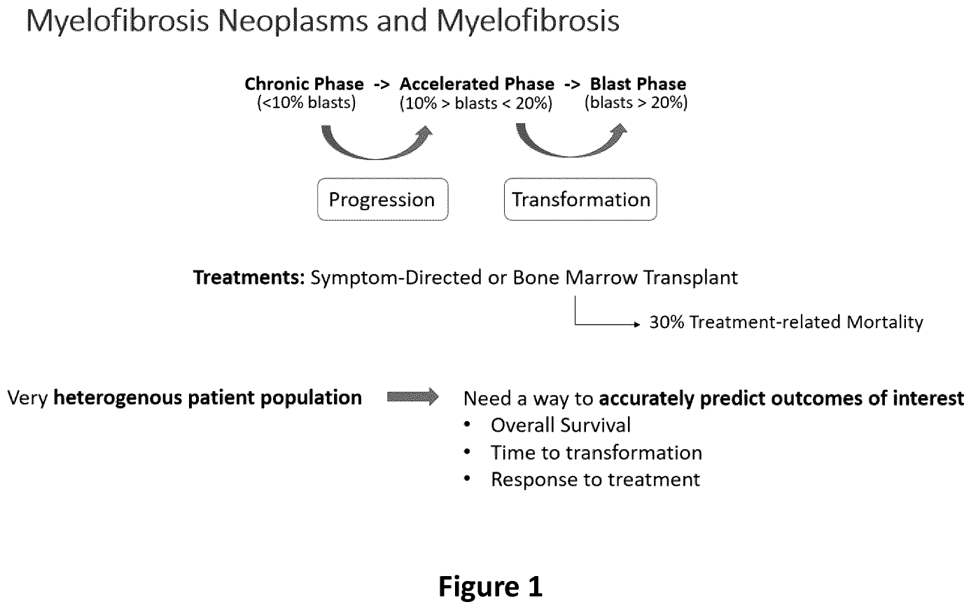

Referring to FIG. 1, myeloproliferative neoplasms (referred to hereafter as “MPN”) represent a collection of chronic, stem-cell driven diseases characterized by the excess production of mature myeloid blood cells and extramedullary hematopoiesis¹. MPNs, including essential thrombocythemia (ET), polycythemia vera (PV), and primary myelofibrosis (PMF), are thought to represent a biological continuum of increasingly severe disease entities^{2,3}. Both ET and PV disease can progress to bone marrow (BM) myelofibrosis (MF), termed post-ET MF and post-PV MF, respectively. While wide variability exists among patients, PMF, post-ET MF and post-PV MF (collectively referred to hereafter as simply “MF”) are at the highest risk for low overall survival (OS), disease progression and leukemic transformation (LT)³. LT from MF to blast-phase acute myeloid leukemia (BP-AML; >20% blasts in the BM and/or peripheral blood (PB)) occurs in >25% of cases. Once transformed, BP-AML is even more refractory to conventional therapy than both de novo or other secondarily derived myeloid leukemias⁵.

While the outcome of disease progression and leukemia transformation are severe risks for individuals with MPN, and magnified in those with MF, current treatments are predominantly palliative, with limited disease modulating effects. These include the use of hydroxyurea as a cytoreductive agent and/or JAK inhibitors, predominantly Ruxolitinib. Allogeneic bone marrow transplantation (BMT) can be curative but is associated with high treatment-related morbidity and mortality (30%) and should be reserved for only the most high-risk individuals, where risk of death from disease exceeds that of the procedure intended to cure it. Therefore, accurate risk stratification is paramount to guide clinical decision making in MF.

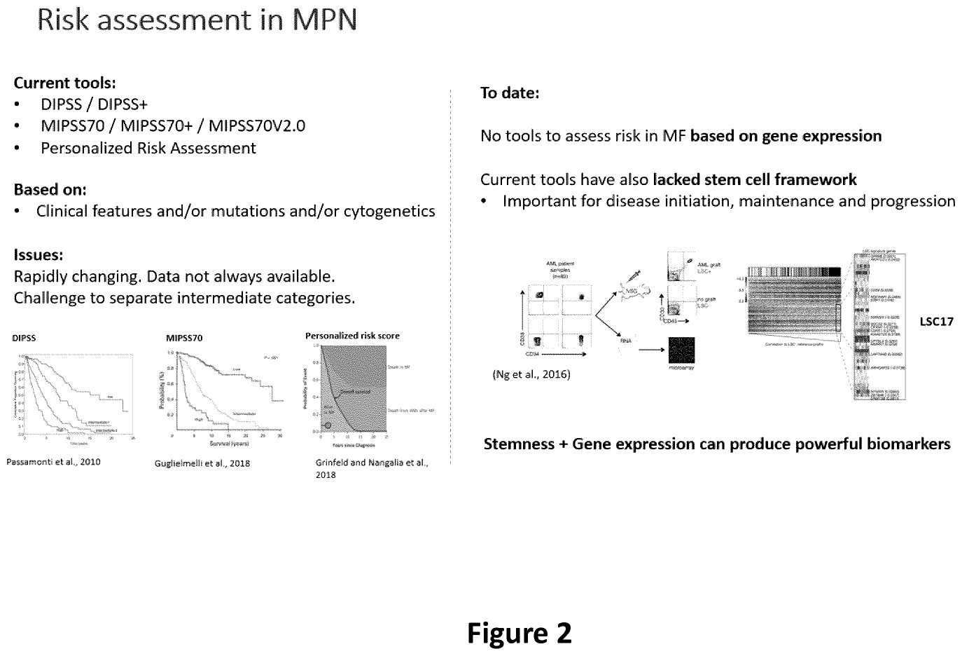

While this need is well recognized, current models built to address it do not consider the properties of the disease driving stem-cell population, and so the picture of disease and our prognostic potential in this setting remains incomplete. For example, referring to FIG. 2, the Dynamic International Prognostic Scoring System (DIPSS) is the most widely used risk stratification model in MPN but relies primarily on clinical features and is entirely devoid of disease-related molecular information that can further help characterize the disease. MIPSS70 aims to address this in part by integrating the use of common somatic mutations in MPN that can impact disease outcomes. While a step forward, current approaches for acquiring the mutational data needed to generate the MIPSS70 score is costly and non-standard, and therefore is not yet widely used in practice. More recent iterations include the use cytogenetics (DIPSS+, MIPSS70v2.0+) but these are used to an even lesser degree for the same reasons as well as increased complexity. Further, many of these iterations are being performed on the same patients and so the additional external cohorts are of value. Beyond this, while clinicians are generally confident in their treatment approach for low and high-risk patients by these methods, the approach for the majority of intermediate classified individuals is more ambiguous and merits further refinement. To date neither of these clinically used scores (DIPSS or MIPSS70) incorporate the use of gene expression which could provide useful, and non-overlapping information. Most critically, to date, no prognostic scores in MPN, account for the properties of the disease-driving stem cell population.

Thus, there is a need for a simple, cost-effective clinically deployable assay for more effective prognostic stratification in MF, that considers the disease-driving stem cell population. In this way, not only will prognostic genes identified serve as powerful biomarkers for disease progression and leukemic transformation but might also represent functional targets that can modulate disease-outcomes. This information may be useful as a stand-alone test or be incorporated with known clinical and/or molecular features currently used to predict disease outcomes.

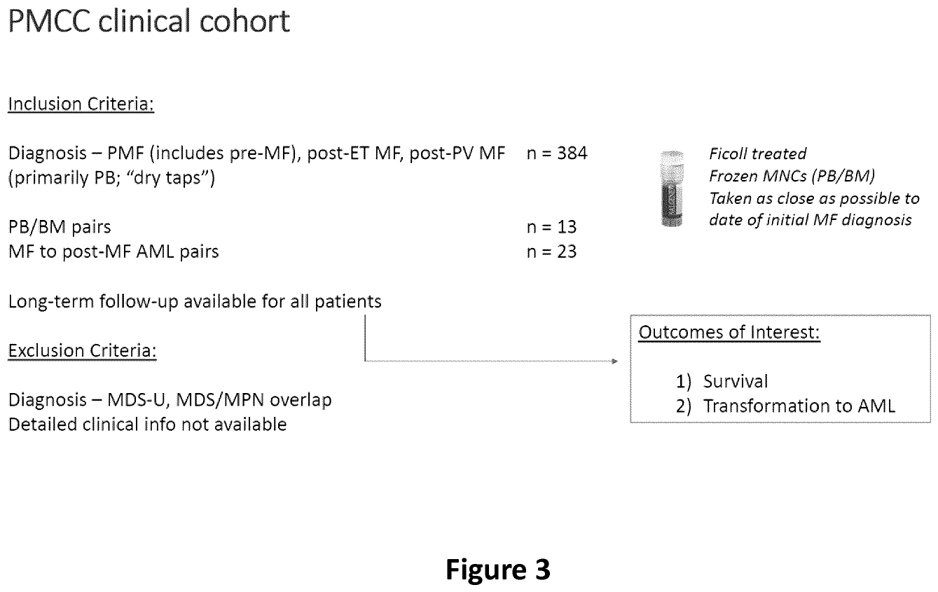

To approach this problem, we built a patient cohort that met our inclusion criteria for MF, contained a biological sample (PB and/or BM), had detailed clinical data across a number of relevant parameters and long-term follow-up with associated outcomes data (survival, leukemic transformation) (see FIG. 3). All patients were seen at Princess Margaret Cancer Center (PMCC).

Our inclusion criteria consisted of individuals with a diagnosis of PMF, pre-fibrotic MF, post-ET MF and post-PV MF. Individuals were excluded if they received a diagnosis of MPN-Unclassified (MPN-U) or MPN/MDS overlap.

Biological samples collected for each patient were frozen, ficoll-treated mononuclear cells (MNCs) from the PB and/or BM collected as close as possible to the initial date of the patients' MF diagnosis. Since most individuals with MF have a fibrotic marrow, bone marrow aspirates are typically unsuccessful and described as “dry taps”. Thus, the vast majority of patient samples were of PB origin.

384 patient samples met the inclusion criteria for our study. 358 of these were of PB origin and were ultimately the samples that were used to derive our prognostic signature.

For 13 of patients, a BM sample collected from the same patient at the same time as the PB samples was collected in order to compare PB/BM pairs.

For 23 of patients, a sAML sample was collected from a MF patient who later transformed to leukemia for comparison between MPN/SAML samples from the same individual.

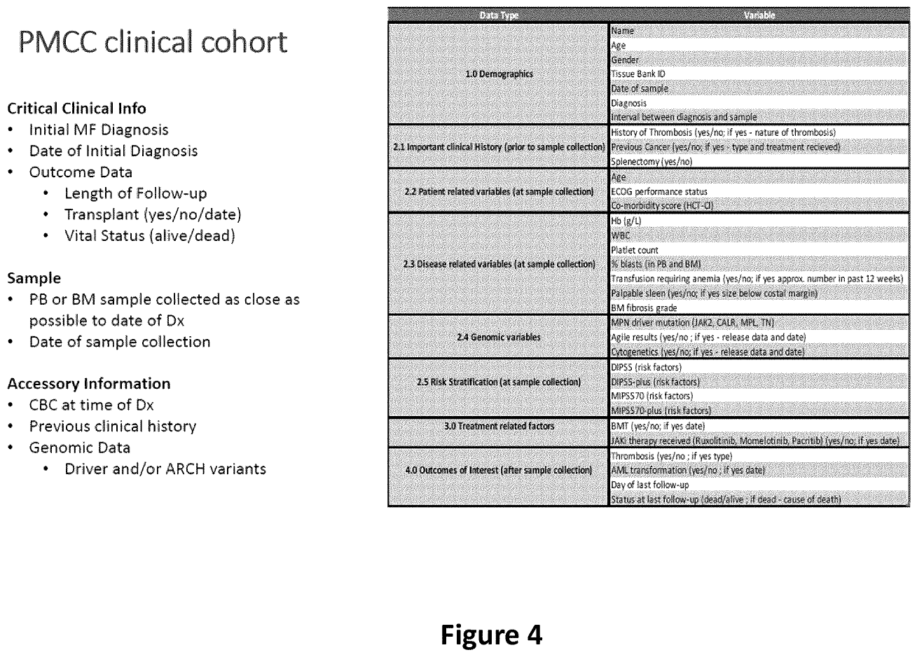

Referring to FIG. 4, data collection parameters were collected for all patients broadly fell into the following categories: demographics, important clinical history (prior to sample collection), patient-related variables (at sample collection), disease-related variables (at sample collection), genomic variables, current risk stratification (at sample collection), treatment-related factors and outcomes of interest.

The most critical clinical variables included:
Diagnosis related variables: initial MF diagnosis and date of initial MF diagnosis.
Outcomes related variables: length of follow-up, transplant (yes/no/date), transformation status (yes/no/date), vital status (dead/alive/date).
Sample related variables: sample source (PB/BM) and date of sample collection.

Features needed to calculate currently used DIPSS and MIPSS70 scores were also collected to allow comparative analysis.

Our cohort had an average age of 64, a transformation rate of 14% with clinical follow-up of up to 12.2 years. Since the transformation rate is less than what is expected in the literature this specific cohort will likely mature with time to acquire additional events related to OS and LT.

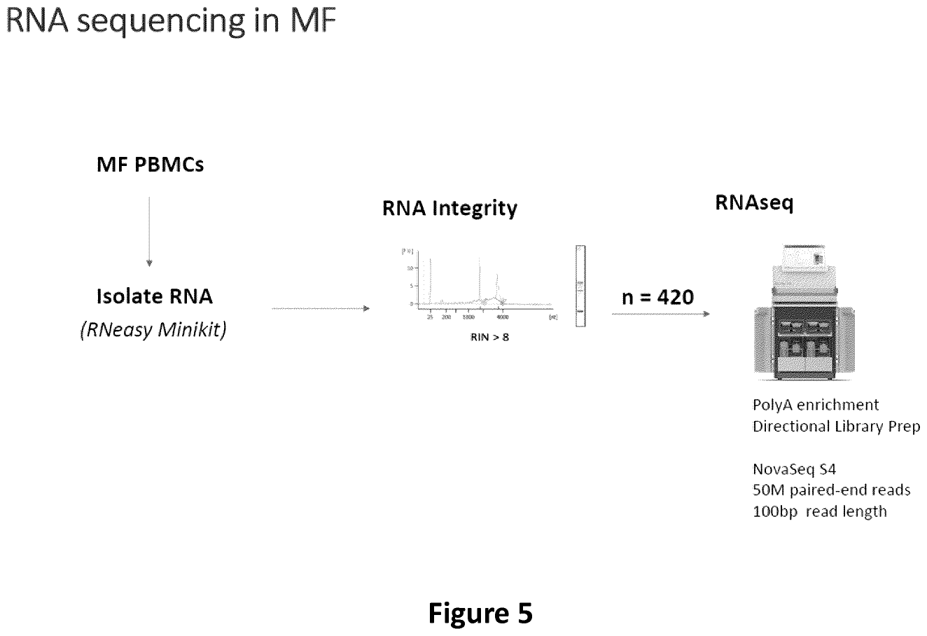

Referring to FIG. 5, RNA was extracted for from unsorted mononuclear cells from all samples that met the inclusion criteria for the study (n=420) using the RNeasy Minikit. RNA with an RNA integrity number (RIN)>8 proceeded to PolyA enrichment and directional library preparation at SickKids Genomic Facility. Libraries were sequenced on the NovaSeq S4 at read-length of 100 bp with approximately 50 million paired-end reads per sample. Sequencing reads were aligned to the reference genome and normalized using standard bioinformatic approaches.

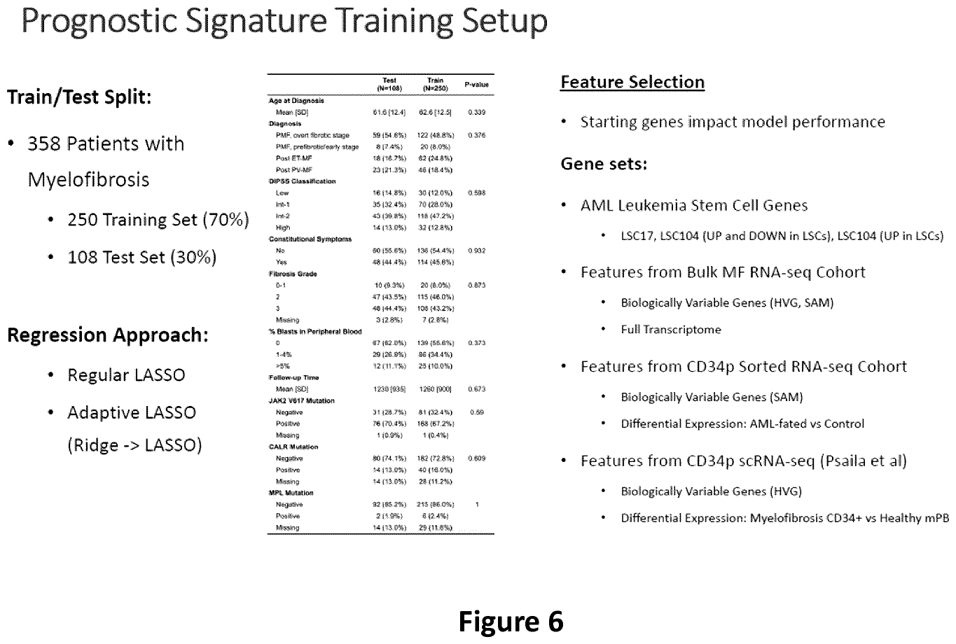

Referring to FIG. 6, we focused only on MF patients for whom we had a PB sample as a biologic (n=358). This is the most relevant sample type since BM taps are typically unsuccessful and PB represents a more easily accessible tissue that is relatively non-invasive and could be done in a longitudinal manner.

To train and validate novel prognostics scores in MF, we randomly split this cohort (n=358) into train (70%; n=250) and test (n=30%; n=108) sets, respectively. We show that there are no statistically significant differences between the train and test sets across multiple clinically relevant parameters.

To train a gene expression score for predicting patient survival, we employed Cox proportional hazards regression using LASSO. LASSO regression is a type of penalized linear regression that deliberately tries to eliminate features/genes in order to find a minimal subset of features/genes that can best predict the outcome of interest (e.g. survival).
For example, if we started with expression of 1000 genes and tried to predict survival using linear regression, it would assign some coefficient to each of the 1000 genes, regardless of whether the genes are associated with survival or not.
However, if we use LASSO, it would assign a coefficient of ZERO to any genes that are not sufficiently informative for predicting survival, eliminating those genes from the final score.
Thus, running LASSO regression with 1000 starting genes may lead to a score of something like 20 genes, wherein 980 non-informative genes were eliminated.
LASSO achieves this by assigning a penalty to larger models, using a parameter called lambda.

Briefly, we ran regular LASSO (described above) alongside variations of Adaptive LASSO, which set custom penalty factors for each gene based on their association with survival (as evaluated by other regression approaches, such as Ridge or Elastic Net). This has been shown in some cases to help LASSO perform better than using the same penalty factor for every gene.

### Feature/Gene Selection

We have learned from LSC17 (Ng et al Nature 2016) that the starting gene set used to train a prognostic score has a profound impact on the performance of the final score, and that starting with a biologically motivated set of genes can produce scores that outperform those generated from starting with the entire transcriptome (e.g. 20,000+ genes). Feature selection is a critical aspect of any machine learning problem and is especially important when we have much more features (>20,000 genes) than samples (250 samples in training set), where risk of overfitting is high.

Thus, we defined 36 distinct starting genesets to train our survival scores from, which are broadly outlined in the slide. The genesets defined ranged from those associated with leukemia stem cells (Ng et al., 2017), the entire transcriptome and highly variable genes from our PMCC bulk RNAseq data, highly variable genes derived from bulk RNAseq on an internal set of CD34+ sorted MF stem cells and highly variable genes from single-cell RNAseq data derived from MF stem cells (Psaila et al., 2020).

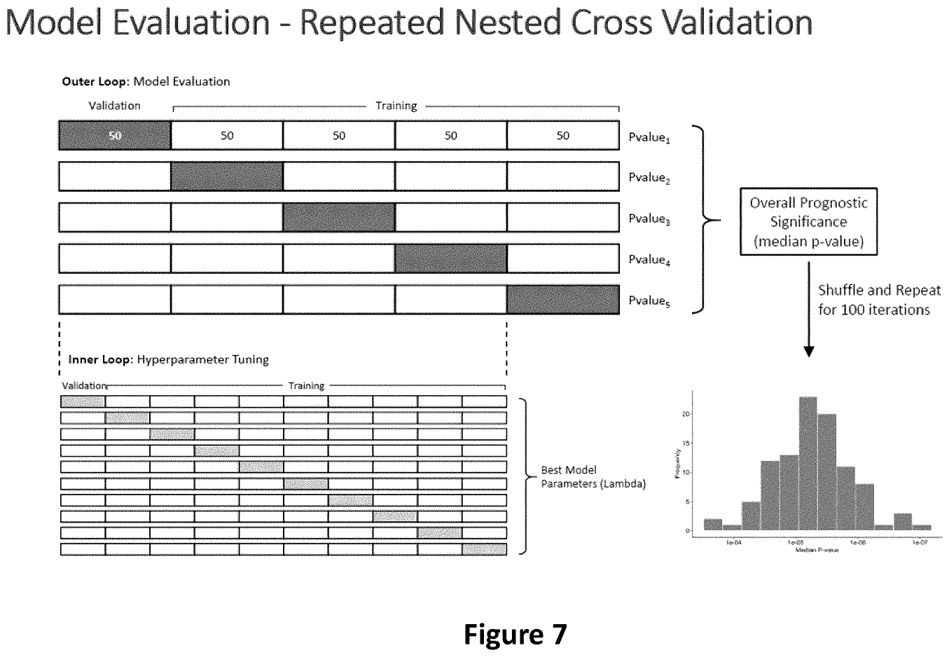

Referring to FIG. 7, we now have 36 starting genesets and at least two regression methods (Regular LASSO and Adaptive LASSO, other variations not shown) to generate survival scores from, and need to identify the best combination of starting gene set+LASSO regression approach for predicting survival in our cohort. We evaluated this through repeated nested cross validation In order to avoid overfitting and memorizing the training set, we always need to evaluate the performance of a model on a separate group of patients that it did not see during model training (e.g. a validation set). Thus, for a given set of starting genes, the 250 training patients were grouped into 5 bins of 50 patients each. Each bin (n=50) was used as the validation set while the remaining bins (n=200) were used to train the model from these starting genes. This results in 5 models, each with a p-value showing their performance in predicting survival on the validation set. The median of these 5 p-values is used to represent the overall performance of this model configuration on data it has not seen before. This is referred to as cross validation.

Each time these models are trained on those 200-patient subsets, there is another cross-validation approach that is used to determine the best penalty parameter for LASSO. This is the internal cross-validation loop. Collectively, this is called nested cross validation.

After each nested cross-validation run, we randomly shuffle the data and re-split the patients, repeating this nested cross-validation process a total of 100 times. This is repeated nested cross-validation, and it results in a distribution of p-values of model performance based on 500 random splits of the data for each combination of starting gene set+LASSO method. This is what we use to identify the most predictive set of genes while minimizing bias from overfitting.

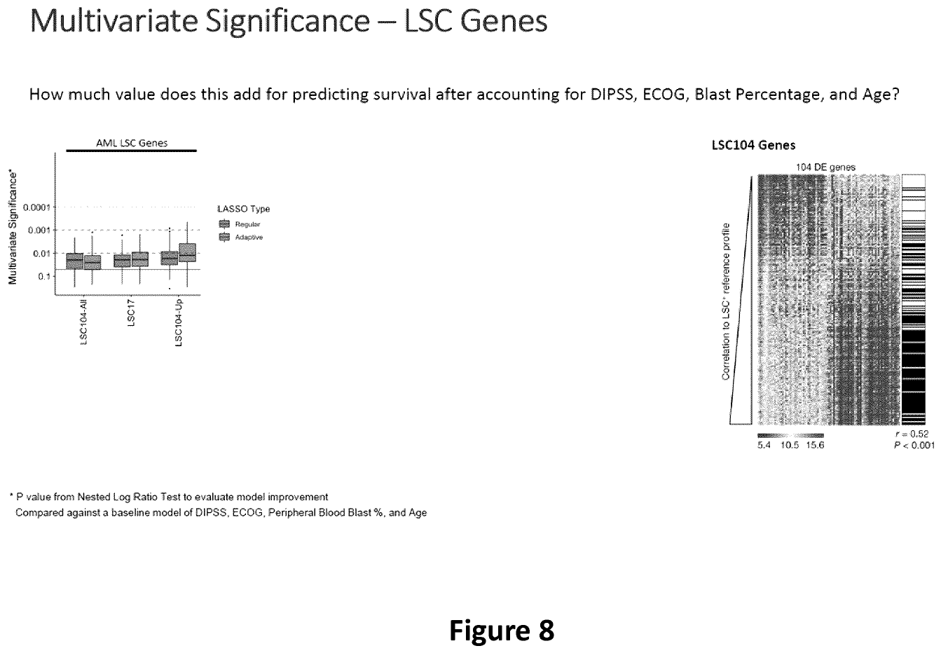

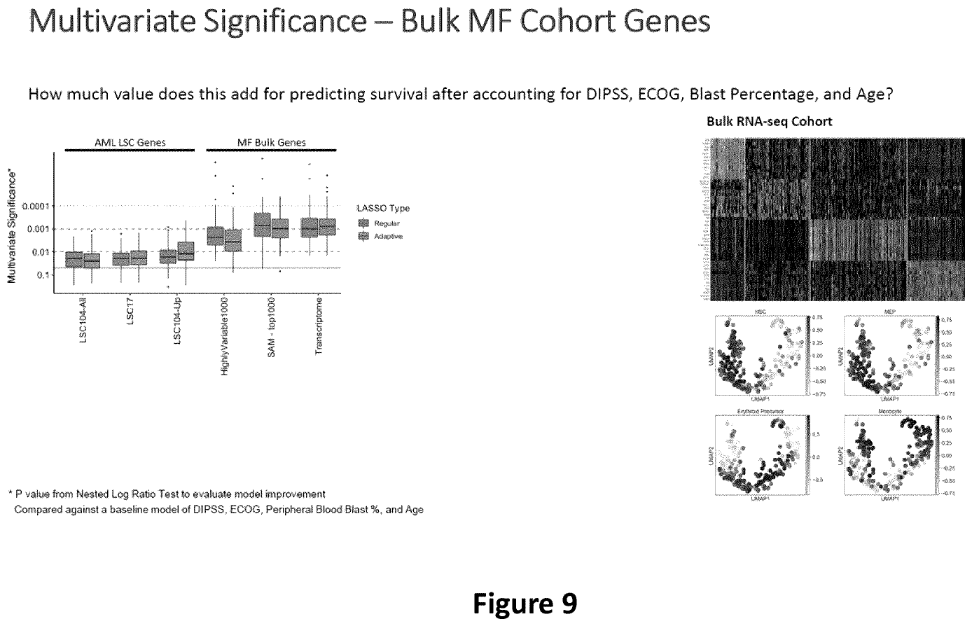

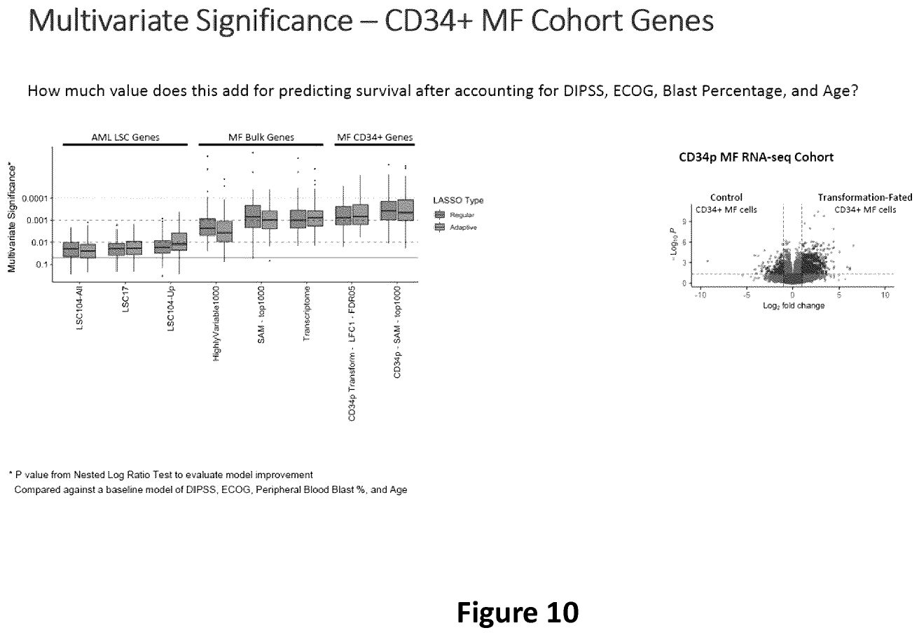

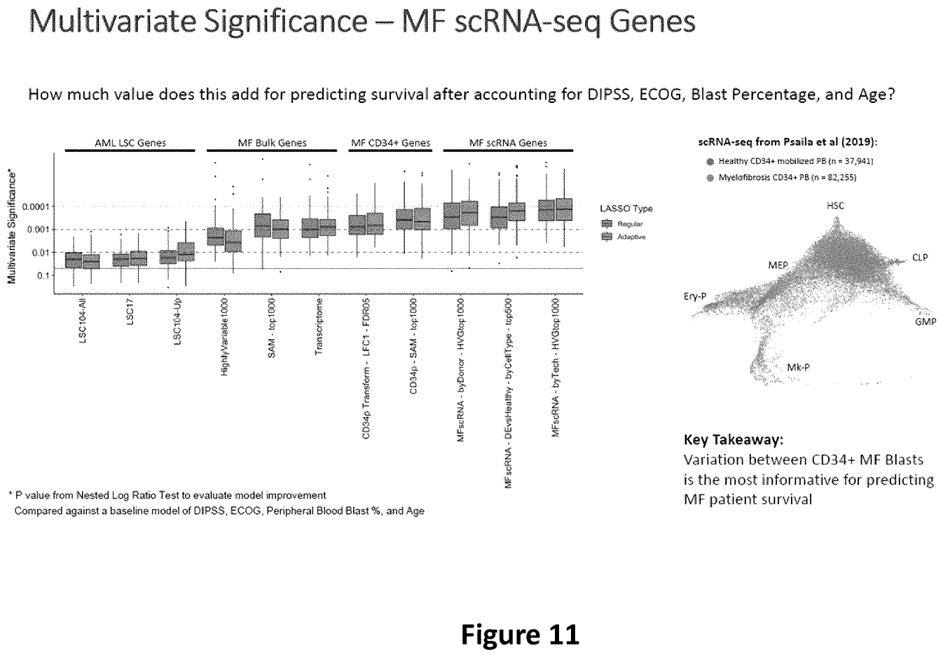

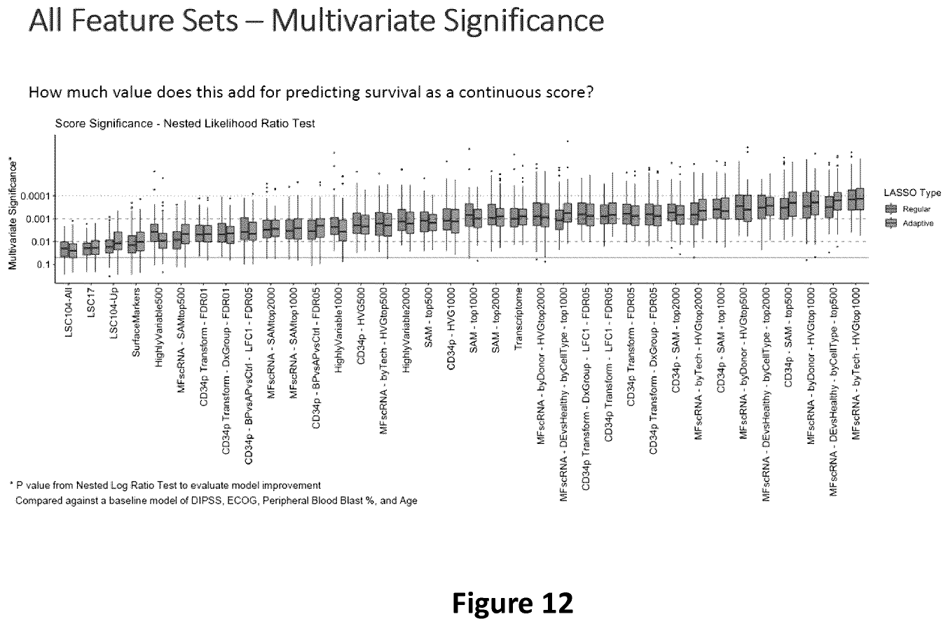

Referring to FIGS. 8-12, we tested 36,000 models in total, derived from 36 initial MF-related genesets. Critically, the most accurate models by cross validation (median multivariable p-value=6e-5) were produced from our retrospective identification of highly variable genes in single-cell RNAseq data derived from 82,255 Lin-CD 34+ MF stem and progenitor cells across 15 patients (Psaila et al., 2020) (specifically, the geneset we defined as “MFscRNA-byTech-HGVtop1000”). Thus, features of intra- and inter-patient heterogeneity among MF stem and progenitor cells proved to be the most relevant for predicting survival.

Note that the multivariable p-values here represent how significantly the scores generated from each starting gene set predicts survival after adjusting for already well-established prognostic factors (DIPSS, ECOG, Peripheral blood blast %, and Age).

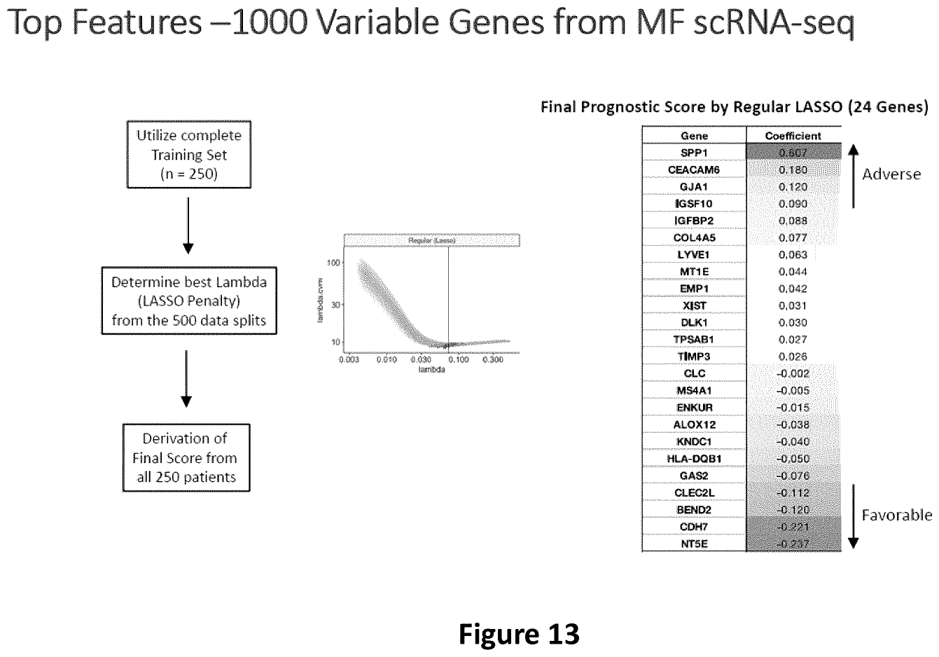

Referring to FIG. 13, from these features (“MFscRNA-byTech-HGVtop1000”), we then used the entire training set (n=250) to derive our final model. We determined the optimal lambda (LASSO penalty) to use by analyzing the inner loop hyperparameter tuning preformed in the 500 data splits from the section above.

This approach generated a final model calculated as the weighted sum of gene expression across 24 genes (termed MPN 24) predictive of overall survival in MF. Importantly, since these genes were derived from disease-propagating MF stem cell populations, they may act as both biomarkers but also potential biological targets in future studies.

Referring to FIG. 14, to evaluate the utility of the MPN24 score to predict OS in MF, we first categorized patients with MPN24 scores above or below the training cohort median as MPN24 high or low, respectively. As expected, MPN24 worked extremely well in the training set (n=250) but is likely overfit since the model was derived on from this set of patients. In this training set, high and low score patients experienced 5-year survival rates of 85% [95% CI 77-94%] and 21% [95% CI 13%-32%], respectively, when censored at time of BMT (HR=11[95% CI 6.3-19]; p<0.0001), when censored at time of BMT. More importantly, MPN24 was validate in the test set (n=108) that was left out of training the model. In the test set, with high and low score patients experiencing 5-year survival rates of 71% [95% CI 57-88%] and 21% [95% CI 9%-52%], respectively, when censored at time of BMT (HR=5.3[95% CI 2.6-10.5]; p=2.1e-6).

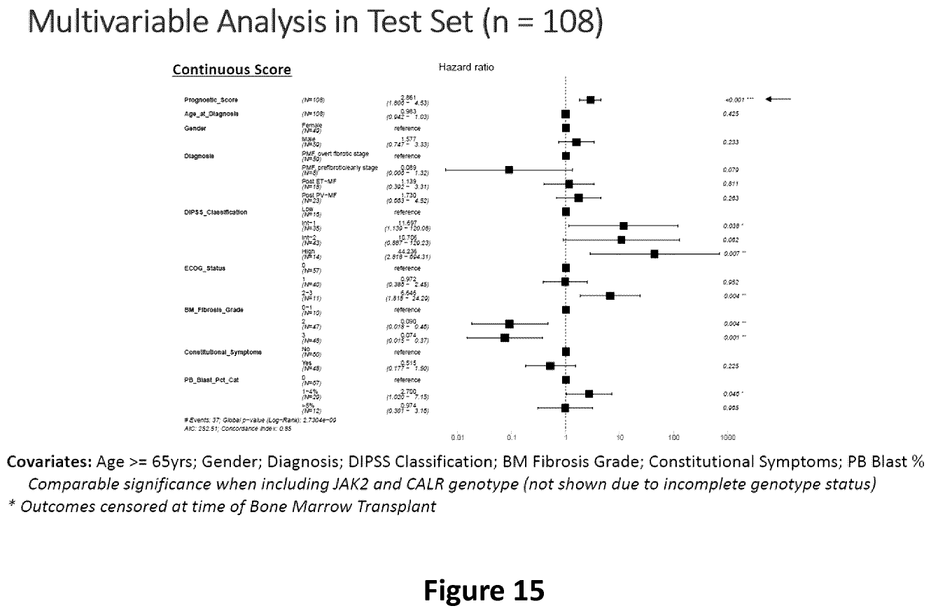

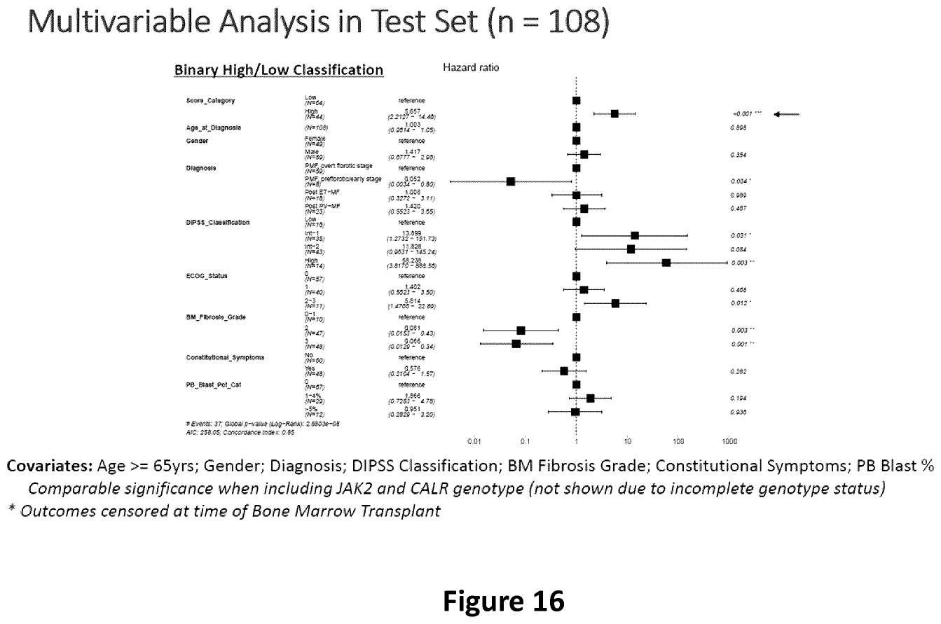

Referring to FIGS. 15 and 16, MPN24 as a continuous variable retained independent prognostic value in multivariable analysis incorporating age, sex, DIPSS category, ECOG status, fibrosis grade, constitutional symptoms, and PB blast percentage (adjusted HR=2.9[95% CI 1.8-4.5]; p=<−0.001). When MPN24 was stratified as a binary as low and high based on the MPN24 scores below or above the median threshold derived in the training set (n=250), respectively, similar results were achieved (adjusted HR=5.7[95% CI 2.2-14.5]; p=3e-4). Importantly, DIPSS classification remained a significant covariate, indicating that MPN24 and DIPSS capture distinct features of disease.

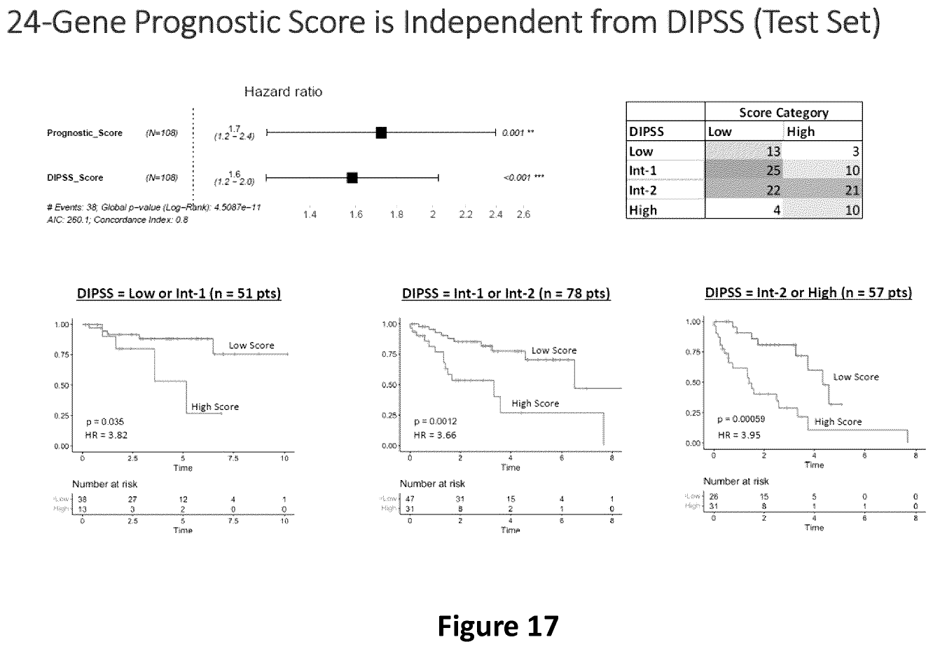

We thus thought that MPN24 might be incorporated with existing DIPSS categorization to generate an augmented risk stratification scheme for MF. Referring to FIG. 17, indeed, in the test set (n=108) MPN24 low and high scores as previously defined, were able to segregate patients previously categorized by DIPSS as low or Intermediate-1 (n=51, HR=3.82; p=0.0035), Intermediate-1 or Intermediate-2(n=78 , HR=3.66; p=0.0012), and Intermediate-2 or high (n=57, HR=3.95; p=0.00059) in a Kaplan-Meier analysis. For this analysis patients in adjacent DIPSS categories had to be binned together due to a small sample sizes once subcategorizing the test set (n=108). In the future, the additional MF patient cohorts will power this analysis sufficiently to treat each DIPSS category independently.

Referring to FIG. 18, since MPN24 and DIPSS captured distinct features of poor outcomes in MF we devised as new 3-tier classification scheme to incorporate the two prognostic models.

Patients were newly classified in this 3 tier model as follows:
Patients classified by DIPSS as Low or Int-1 and MPN24-low were newly classified as “Low” (n=38)
Patients classified by DIPSS as Low or Int-1 but MPN24-high as well as patients classified by DIPSS as Int-2 or High but MPN24-low were newly classified as “Intermediate” (n=39)
Patients classified by DIPSS as Int-2 or High and MPN24-high were newly classified as “High” (n=31)

With his new integrated model there was more equal partitioning of patients into the 3 different risk classifications compared to the DIPSS score where the vast majority of patients were classified in the Intermediate 1 and 2 categories where clinical decision making remains ambiguous. When categorized by DIPSS, 14 (13%), 43 (40%), 35 (32%) and 16 (15%) patients fell into High, Int-2, Int-1, and Low categories, respectively. When DIPSS and MPN24 were integrated, 31 (29%), 39 (36%) and 38 (35%) patients fell into High, Intermediate, and Low categories, respectively. Patients classified as low-, intermediate- or high-risk in this new classification scheme experienced 5-year survival rates of 88.2% [95% CI 77.9%-99.9%], 39.3% [95% CI 19.9%-77.7%] and 10.8% [95% CI 2.1%-55.8%], respectively (likelihood ratio test p=1e-8).

Referring to FIG. 19, similarly tiering was performed with MPN24 integrated with MIPSS70 score. We collected the genomic data required to run the MIPSS70 score on patients in our test set (n=78) and show that MPN24 can further stratify MIPSS70 defined intermediate and high risk groups. Given that leukemic transformation is a negative contributor to overall survival we hypothesized that genes in the MPN24 might also capture features specific to leukemic transformation in MF. Therefore, referring to FIG. 20, we set out to retrain the MPN24 to derived a new gene subscore predictive of leukemic transformation. Beginning with only the genes in the MPN24 score we regressed on time-to-transformation (censoring all other events) within 5 years of sample collection. This cutoff was set because we are specifically interested in individuals who will transform to sAML relatively soon after a sample is collected. Our approach generated a 13 gene subscore (MPN13) predictive of leukemic transformation in the training set (n=250).

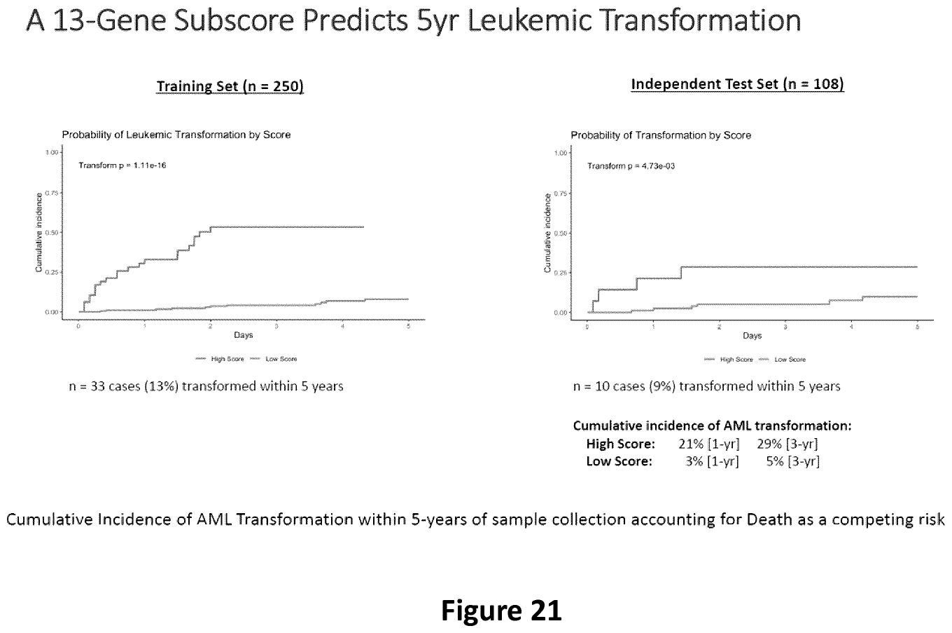

Referring to FIG. 21, since total leukemic sample were limiting, patients scoring above the 80th percentile from the training set (n=250) were classified as high risk and the remaining as low risk. In the training set, 33 (13%) individuals transformed to sAML within 5 years while 10 cases (9%) transformed in the test set. Still, MPN 13 was significantly associated with risk of transformation (p=1.1e-16) in the train set. More importantly, MPN13 was validated in the test set (p=4.7e-03) with low and high-risk patients experiencing 3-year cumulative incidences of transformation of 5.2% [95% CI 0.2%-10.2%] and 28.6% [95% CI 3.1%-54.0%] respectively, after adjusting for death as a competing risk. In the future, additional patients and external cohorts will allow us to power our analysis for potential integration of MPN13 with DIPSS and/or MIPSS70 as described above for MPN24.

In summary, we have utilized sc-RNAseq data from biologically relevant MF stem cells together with sophisticated machine learning approaches to derive a 24-gene gene expression signature (MPN24) predictive of overall survival and 13-gene subscore (MPN13) predictive of leukemic transformation in MF. MPN24 can be used alone or be integrated with currently used clinical risk stratification models to more appropriately assess risk in MF. We predict that these scores will be used in the clinic to better inform patient management, particularly in the context of BMT or experimental drugs in the context of clinical trials.

To quantify the MPN24 signature we deployed a NanoString-based approach to profile the 24 corresponding genes. Further, an in-house analysis of our bulk RNAseq data, identified an additional 24 independent genes to serves as our “reference genes”. These genes were selected to span a similar range of expression levels as those in the MPN24 signature, while simultaneously having narrow variance across multiple samples. Both the MPN24 and reference genes were submitted to NanoString Technologies for custom CodeSet creation using nCounter Elements TagSets. All Probe A and Probe B oligos designed were procured from Integrated DNA Technologies using standard desalting and PAGE purification, respectively. Probe A and B Master Stocks were prepared according to the manufacturer's instructions.

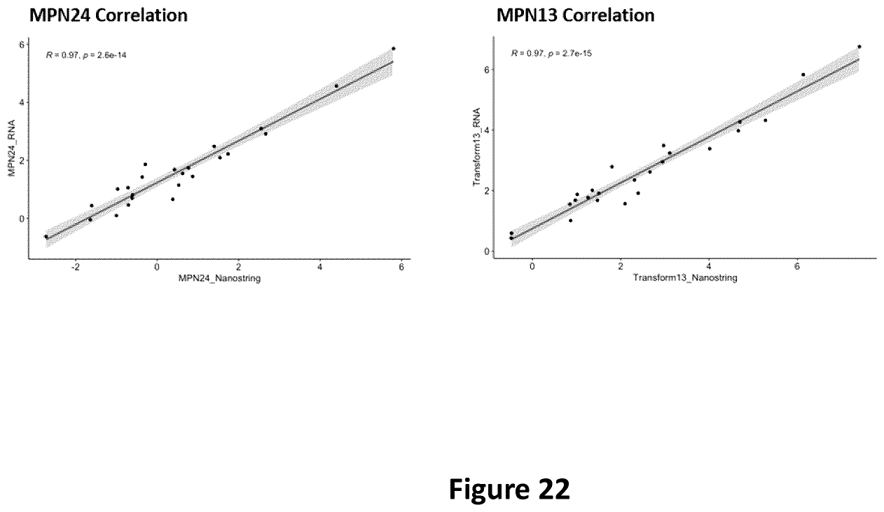

Referring to FIG. 22, the MPN13 and MPN24 signatures were measured using Nanostring and showed high correlation to RNAseq—0.97 with high significance. The NanoString assay was performed by Princess Margaret's core sequencing facility according to the manufacturer's protocol. Briefly, 100 ng of total RNA isolated from peripheral blood mononuclear cells were submitted at 20 ng/ul. Quality of RNA was confirmed via Eukaryote Total RNA Nano BioAnalyzer assay using 2100 Expert Software (Agilent). Elements hybridization reactions were completed using 5 ul of RNA per sample (100 ng) incubated with Probe A mix, Probe B mix and Elements TagSets at 67° C. for 16-48 hours to create complete Tag Complexes on the nCounter Prep Station (version 4.0.11.1). After hybridization, excess probes were washed out using a 2-step magnetic bead-based purification strategy according to the manufacturer's protocol, and purified target/probe complexes (i.e. Tag Complexes) were immobilized on the NanoString cartridge for data collection. Transcript counts were determined using the nCounter Digital Analyzer (version 2.1.2.3) at the high-resolution setting. Specifically, digital images were processed with final barcode counts tabulated in reporter code count (RCC) output files.

RCC output files were loaded into the nSolver software, wherein mRNA transcript abundance values of genes comprising the MPN24 signature were normalized according to the geometric mean of either the housekeeping “reference genes”, the positive spike-in control transcripts, or both sets of controls. Normalized transcript abundance values of each MPN24 gene were subsequently multiplied by the weight of each component gene within the MPN24 and MPN13 equations, and the sum of these values were used to represent the MPN24 and MPN13 scores for each patient, respectively.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

### REFERENCE LIST

1. Tefferi A, Pardanani A. Myeloproliferative neoplasms: A contemporary review. JAMA Oncol. 2015; 1(1):97-105. doi:10.1001/jamaoncol.2015.89
2. Tefferi A. Novel mutations and their functional and clinical relevance in myeloproliferative neoplasms: JAK2, MPL, TET2, ASXL1, CBL, IDH and IKZF1. Leukemia. 2010; 24(6):1128-1138. doi:10.1038/leu.2010.69
3. Vannucchi A M, Harrison C N. Emerging treatments for classical myeloproliferative neoplasms. Blood. 2017; 129(6):693-703. doi:10.1182/blood-2016-10-695965
4. Cerquozzi S, Tefferi A. Blast transformation and fibrotic progression in polycythemia vera and essential thrombocythemia: A literature review of incidence and risk factors. Blood Cancer J. 2015; 5(11):e366-10. doi:10.1038/bcj.2015.95
5. Østgård LSG, Medeiros B C, Sengeløv H, et al. Epidemiology and clinical significance of secondary and therapy-related acute myeloid leukemia: A national population-based cohort study. J Clin Oncol. 2015; 33(31):3641-3649. doi:10.1200/JCO.2014.60.0890.

1. A method of prognosing or classifying a subject with a Myeloproliferative Neoplasm (MPN) comprising:
(a) determining the expression level of at least 10 genes in a test sample from the subject selected from the group consisting of SPP1, CEACAM6, GJA1, IGSF10, IGFBP2, COL4A5, LYVE1, MT1E, EMP1, XIST, DLK1, TPSAB1, TIMP3, CLC, MS4A1, ENKUR, ALOX12, KNDC1, HLA-DQB1, GAS2, CLEC2L, BEND2, CDH7, and NT5E; and
(b) comparing expression of the at least 10 genes in the test sample with reference expression levels of the at least 10 genes from control samples from a reference cohort of patients;
wherein a difference or similarity in the expression of the at least 10 genes in the test sample and the reference expression levels is used to prognose or classify the subject with MPN into a low risk group or a high risk group for worse survival.

2. The method of claim 1, wherein the at least 10 genes is at least 11, 12, 13, 14, 15, 16, 17, 18, 19, 20, 21, 22, 23, or 24 genes.

3. The method of claim 2, wherein the at least 10 genes consists of all 24 genes.

4. The method of claim 1, further comprising building a subject gene expression (GE) profile from the determined expression of the at least 10 genes.

5. The method of claim 4, further comprising obtaining a reference GE profile associated with a prognosis, wherein the subject GE profile and the gene reference expression profile each have values representing the expression level of the at least 10 genes.

6. The method of claim 1, further comprising calculating a MPN Score comprising the weighted sum expression of the at least 10 genes.

7. The method of claim 6, wherein classification of the subject into a high risk group is based on a high MPN24 Score in reference to the control cohort of MPN patients.

8. The method of claim 6, wherein classification of the subject into a high risk group or low risk group is based on whether the subject MPN24 Score is above or below, respectively, a predetermined threshold MPN24 Score of the reference cohort, preferably the mean or median MPN24Score.

9. The method of claim 1, wherein determining the GE level comprises use of RNAseq, quantitative PCR or an array.

10. The method of claim 1, wherein determining the GE level comprises use of nanostring.

11. The method of claim 1, further comprising stratifying the patients based on one or more further criteria selected from sex, DIPSS category, ECOG status, fibrosis grade. constitutional symptoms, MIPSS70 category, or PB blast percentage.

12. (canceled)

13. The method of claim 1, wherein the MPN is Myelofibrosis (MF).

14. The method of claim 1, wherein the MPN is Polycythemia Vera (PV).

15. The method of claim 1, wherein the MPN is Essential Thrombocythemia (ET).

16. The method of claim 1, wherein the MPN is Chronic Myelogenous Leukemia (CML).

17. The method of claim 1, wherein the at least 10 genes are selected based on the most highly correlated genes based on the coefficients in FIG. 20.

18. The method of claim 1, further comprising treating the subject with more aggressive therapy if the subject has been determined to be in the high risk group for worse survival.

19. The method of claim 18, wherein the more aggressive therapy is bone marrow transplant.

20. The method of claim 18, wherein the more aggressive therapy is adjuvant therapy, intensified chemotherapy or an alternative therapy through enrollment into a clinical trial for a novel therapy.

21. A method of determining the risk of transformation of a Myeloproliferative Neoplasm (MPN) to acute myeloid leukemia, in a subject with MPN, comprising:
(a) determining the expression level of at least 5 genes in a test sample from the subject selected from the group consisting of SPP1, TPSAB1, COL4A5, CEACAM6, IGFBP2, EMP1, DLK1, IGSF10, HLA-DQB1, KNDC1, CLEC2L, CDH7, or ENKUR; and
(b) comparing expression of the at least 5 genes in the test sample with reference expression levels of the at least 5 genes from control samples from a reference cohort of patients;
wherein a difference or similarity in the expression of the at least 5 genes in the test sample and the reference expression levels is used to prognose or classify the subject with MPN into a low risk group or a high risk group for transformation to secondary acute myeloid leukemia (sAML).

22-40. (canceled)

41. A computer program product for use in conjunction with a computer having a processor and a memory connected to the processor, the computer program product comprising a computer readable storage medium having a computer mechanism encoded thereon, wherein the computer program mechanism may be loaded into the memory of the computer and cause the computer to carry out the method of claim 1.

42. A kit comprising reagents for detecting the expression of the genes defined in claim 1.

43. An array or nanostring for detecting the expression of the genes defined in claim 1.
